(19) United States
(12) Patent Application Publication
(10) Pub. No.: US 2025/0078996 A1
(43) Pub. Date: Mar. 6, 2025

(54) APPARATUS AND METHOD FOR PROVIDING TELEMEDICINE SERVICE

(71) Applicant: FLYINGDOCTOR INC., Goyang-si (KR)

(72) Inventor: Woo Jin LEE, Seoul (KR)

(21) Appl. No.: 18/724,094

(22) PCT Filed: Dec. 30, 2022
(86) PCT No.: PCT/KR2022/021703

§ 371 Date: Jun. 25, 2024

(30) Foreign Application Priority Data
Dec. 30, 2021 (KR) 10-2021-0192296
Dec. 29, 2022 (KR) 10-2022-0189646

(51) Int. Cl. G16H 40/67

(52) U.S. Cl. CPC G16H 40/67

(57) ABSTRACT

An apparatus for providing a telemedicine service is presented. The apparatus may be a service provision server comprising a communication interface and a processor. According to an embodiment, when a patient client associated with a patient terminal completes a medical treatment registration and requests access to a medical office, the patient client is admitted to the medical office. In addition, when the patient client has accessed the medical office, video link access of a doctor client associated with a doctor terminal is processed to start a video link between the doctor client and the patient client.

### TECHNICAL FIELD

The following description relates to an apparatus and method of providing a telemedicine service.

### BACKGROUND ART

Telemedicine utilizes telecommunications and information technology to provide clinical health care from a distance. Telemedicine eliminates the barriers of distance and makes medical services more accessible. In many cases, telemedicine is used in rural areas where continuous medical services are challenging due to distance. Telemedicine is also used to save lives in critical care or emergency situations. Recently, telemedicine has been permitted on a limited basis for the consultation of chronic diseases and/or follow-up patients to prevent infectious diseases.
Korean Patent Publication No. 10-0844543 (registered on Jul. 1, 2008) discloses a method and apparatus for implementing a web-based remote diagnosis.
Korean Patent Publication No. 10-0366816 (registered on Dec. 18, 2002) discloses a telemedicine apparatus and a method thereof.

### DISCLOSURE OF THE INVENTION

### Technical Goals

In providing a telemedicine service, the process of telemedicine is optimized to improve the efficiency of a medical consultation. Like an in-person consultation at a hospital, when a health care provider is ready to start a consultation, a patient is prepared in advance. This ensures that a health care provider, who needs to see many patients, does not waste unnecessary time.

Traditional services typically involve a health care provider contacting a patient after an appointment is scheduled, but without a precise appointment time. In such a case, the health care provider needs to wait for the patient to connect with health care provider, and when the patient misses the connection attempt, the health care provider has to repeatedly try to reconnect with the patient. This may also be problematic for the patient, as the patient may receive repeated contact attempts at unexpected times. By preventing these inconveniences and inefficiencies, telemedicine may proceed smoothly for both parties.

Additionally, a patient may also use a telemedicine service efficiently without experiencing long wait times during the process. This also enhances the patients' or clients' focus on the content being provided.

The order of consultations for patients visiting a hospital and patients accessing a telemedicine service is optimized.

### Technical Solutions

Ensuring a Health Care Provider's Immediate Availability for a Consultation by Confirming that a Patient is Waiting in a Virtual Consultation Room

According to an embodiment, an apparatus for providing a telemedicine service is disclosed. The apparatus may include a communication interface and a processor. The apparatus may include at least one computing device such as a server-class computer, a workstation, a desktop computer, a laptop computer, or a mobile electronic device. The processor may perform the following operations by processing computations related to installed and running software. The processor may admit a patient client to a virtual consultation room when the patient client associated with a patient terminal completes consultation registration and requests access to the virtual consultation room. The apparatus may be connected to a health care provider client, which is an application installed on a hospital and/or health care provider terminal, as well as to the patient client, which is an application installed on the patient terminal, through a communication interface. The application may be original application software separately installed on a terminal or a web page, which is a web-based page accessed through a browser and replaces an app.

In the case of an in-person consultation at a hospital, a patient waits in a waiting area until the patient's turn and then enters a consultation room, ensuring that a health care provider may start a consultation without delay. However, for telemedicine, traditional services often involve a patient applying for an online consultation, after which a health care provider (or hospital) attempts to connect with the patient. Such a connection may be via a video call, a voice call, and the like. However, since telemedicine involves an online connection rather than a face-to-face meeting, a patient may engage in other activities or leave a terminal unattended while waiting for the consultation. This results in the inconvenience of not guaranteeing immediate availability when the health care provider (or hospital) is ready to start the consultation. According to an embodiment, the processor may check through the patient client whether the patient is actually prepared for the consultation and ensure that an immediate consultation may start as soon as the health care provider starts the procedure. This information may be made available to the health care provider and/or hospital client. According to an embodiment, the processor may process a video connection for the health care provider client associated with a health care provider terminal when the patient client is connected to a virtual consultation room and maintains that connection.

This ensures that a health care provider or hospital may request to start the consultation knowing that the patient is prepared for telemedicine. As a result, a health care provider and a hospital, who need to see many patients, do not experience connection delays or failures. In other words, telemedicine guarantees the possibility of a health care provider starting a consultation with a patient immediately, thereby contributing to improved consultation efficiency for both a hospital and a health care provider.

### Monitoring Connection Maintenance Through a Patient Client

According to an embodiment, the processor repeatedly checks whether the patient client maintains the connection to the virtual consultation room and provides the information on whether the patient client maintains the connection to the health care provider client. The health care provider may then request to start the consultation for the patient who maintains the connection. This reduces the likelihood that the connection with the patient fails and the consultation may not start when the health care provider is ready to start the consultation.

For example, but without being limited thereto, the processor may use HTML5-based WebSocket to exchange a signal with the patient client through the communication interface to monitor the maintenance of the connection of the patient client. The processor may determine whether the connection is maintained based on whether the patient client provides a predetermined response in the virtual consultation room.

### Confirming the Possibility of Starting a Consultation by Checking Whether a Patient Stares at a Camera of a Terminal

According to an embodiment, the processor may check through the patient client whether a person who is staring at a camera associated with the patient client is identified at the camera and provides a verification result to the health care provider client. Typically, the patient client may be a personal device such as a smartphone, a tablet, and a personal computer (PC) of the patient. Therefore, when someone is staring at a camera of a corresponding device, it is highly likely that the patient is ready and waiting for the consultation to start. Thus, in this embodiment, the processor may continuously check through the patient client whether a person who is staring at the camera associated with the patient terminal is identified in the camera.

For example, but without being limited thereto, the processor may utilize a MobileNets model. This model may be used in an embedded vision app, may be based on a depth-wise separable convolution structure, and may have two simple hyper-parameters. These two hyper-parameters may be appropriately selected based on the environment in which the hyper-parameters are used, and the size of the hyper-parameters may also be selectively applied.

For example, the processor may continuously check whether a face is being recognized by the camera of the patient terminal associated with the patient client using the face recognition open-source, face-application programming interface (API), which uses the MobileNets ver. 1 model. The result of this check may be provided as a value between 0 and 1, with values closer to 1 indicating that a face is being recognized. According to an embodiment, when a predetermined threshold is set and the result value is equal to or greater than this threshold, it may be determined that the patient is staring at the camera in the virtual consultation room of the patient client and waiting for the consultation. If the result value remains below the threshold for a predetermined amount of time, such as more than 5 seconds, it may be determined that the patient is not waiting in the virtual consultation room of the patient client. This determination may be provided to the hospital or health care provider client, allowing the health care provider and the hospital to know that the patient is not waiting in the virtual consultation room. Thus, the health care provider may avoid attempting to connect (requesting to start the consultation) with a patient who is not waiting and unlikely to be immediately available for the consultation.

According to an embodiment, when the determined value is less than the predetermined threshold, a message may be provided to the patient through the patient client, instructing the patient to look at the screen and wait for the consultation.

### Event Monitoring Through a Patient Client

When a health care provider intends to start a telemedicine consultation, other embodiments are provided to ensure that a patient is ready. According to an embodiment, the processor may provide content on the virtual consultation room screen when the patient client maintains the connection to the virtual consultation room.

The content may be media content selected by the health care provider or hospital. For example, the content may include YouTube videos featuring the corresponding health care provider, promotional photos or videos about the hospital or health care provider, informational content about diseases or treatment, and advertisements for medical devices, supplies, or pharmaceuticals. However, these examples are only examples, and it shall not be construed as being limited to the examples.

According to an embodiment, it may be detected whether an event of the patient interfacing with the provided content occurs through the patient client, and the detection result may be provided to the health care provider client. For example, but without being limited thereto, the event may be a user input corresponding to at least one of an input of stopping provision of the content (closing), a playback control input for the content (for example, playing, pausing, or seeking forward/backward), or an input of selecting an additional link related to the content (such as playing a recommended video, clicking/tapping a purchase link for an advertised product, and the like). The event may be registered with an event listener and may be selectively added or removed.

### Monitoring Patient's Readiness for a Consultation

When a health care provider intends to start a telemedicine consultation, additional embodiments are provided to ensure that a patient is ready. According to an embodiment, when the patient client maintains the connection to the virtual consultation room, the processor may transmit, to the patient terminal through the patient client, an input request asking for at least one of a pain scale score (visual analogue scale (VAS)), medication side effect information, a response to a survey related to the past consultations, a question the patient wants to ask the health care provider, a photo of an affected area of the patient, or a description of a symptom the patient has.

The fact that a user is entering information in response to this request may be a means for confirming that the patient is waiting in the virtual consultation room for the consultation to start. Additionally, by obtaining information in advance that the health care provider may typically gather through questioning after the telemedicine consultation starts, it may help reduce consultation time and improve the quality of the consultation. According to an embodiment, the processor may allow the health care provider to know whether the patient is entering the requested information (i.e., whether the patient is ready for the consultation) and provide a quantitative assessment of how much of the necessary information is entered to the health care provider client. When multiple patients are entering information in response to the request, the health care provider may select to start a consultation with a patient who is ready first. Even when the information entry is not entirely completed, when the health care provider requests to start a consultation with a patient who is in the process of entering information, it may be ensured with a high probability that the health care provider may have a possibility of starting a consultation immediately.

### Evaluating Readiness for a Waiting Patient and Providing a Possibility of Selecting a Target to be Treated

According to an embodiment, when a plurality of patient clients maintains the connection to the virtual consultation room simultaneously, the processor may evaluate the readiness of each of the patient clients and provide the results to the health care provider client. In actual medical settings, most patients who register for a consultation first or have earlier appointment times are seen by the health care provider first. However, in the case of online consultation, a patient who registers first and is waiting may not always be ready for the consultation. Therefore, in this embodiment, the apparatus for providing a telemedicine service does not process a patient consultation on a first-in first-out (FIFO) basis. Instead, the apparatus enables the health care provider or hospital to identify patients who are ready or complete the necessary steps for the consultation first, thereby enhancing consultation efficiency.

According to an embodiment, the process by which the processor evaluates the readiness of individual patient clients may be based on scoring various evaluation criteria. For example, the readiness of a first patient client may be determined using at least one piece of information of whether a person, who stares at a camera associated with the first patient client, is recognized by the first patient client, whether the requested input information for the patient client is completed, a priority according to a predetermined consultation priority for the first patient client, consultation compliance evaluated based on an average consultation time for the first patient client, or whether the first patient client has a chronic illness.

### Informing a Waiting Patient of an Estimated Wait Time

According to an embodiment, when admitting the patient client into the virtual consultation room, the processor may provide an estimated wait time. This allows a patient waiting for a telemedicine consultation to have an idea of how long the patient will need to wait. Embodiments ensure that whenever the health care provider is ready to start the consultation, the patient may immediately face the health care provider via the screen. Therefore, the goal is not to encourage the patient client to exit the patient client or switch the patient client to another screen during the estimated wait time. Instead, the patient is able to predict the wait time while remaining focused on the patient client in a virtual consultation room. In this process, content may be provided through the patient client described above. The patient needs to stay in the virtual consultation room screen to ensure that the consultation starts as soon as the health care provider is ready, thereby maintaining the patient's focus on the content.

According to an embodiment, when calculating the estimated wait time, the processor may use at least one of the number of other patient clients who have already entered the virtual consultation room of the same health care provider or the number of patients waiting within the hospital for face-to-face consultation with the health care provider.

In this case, the estimated wait time may reflect the sum of a first time, which is the product of a previous average telemedicine consultation time of the health care provider and the number of patients currently waiting for telemedicine consultation, and a second time, which is the product of a previous average consultation time of the health care provider and the number of patients currently waiting in the hospital for the consultation by the health care provider.

### Effects of the Invention

According to embodiments, optimizing the process in providing a telemedicine service may increase consultation efficiency. Similar to an in-person consultation at a hospital, ensuring that a patient is ready for telemedicine when a health care provider is about to start a consultation prevents unnecessary waste of time for the health care provider who needs to see many patients.

According to conventional services, since a medical staff contacts a patient without a precise appointment time after the consultation reservation of the patient, there is a need for the medical staff to spend time or wait to connect with the patient. When the patient misses the connection attempt, the medical staff has to repeatedly attempt to reconnect with the patient. This may cause issues for the patient as well, such as receiving repeated contacts at unexpected times. According to embodiments, such inconveniences and inefficiencies are prevented, ensuring that a telemedicine consultation may be conducted smoothly from both sides.

According to embodiments, a patient using a telemedicine service may utilize the service efficiently without experiencing long and boring wait times during a consultation procedure. A patient may enter necessary information in advance, obtain useful medical information, or gather other details about a hospital and medical staff, making the process time-efficient and providing a user experience similar to that of offline in-person consultation. During this process, a patient waits in a virtual consultation room and keeps an eye on a screen, which ensures that a patient or a customer has a high level of focus on the content provided on the virtual consultation room screen.

Additionally, according to embodiments, the order of consultations for patients visiting a hospital in person and patients accessing a telemedicine service is optimized. This helps in the customer management and administration of a hospital that offers both online and offline consultations.

### BRIEF DESCRIPTION OF DRAWINGS

FIG. 1 is a diagram illustrating a connection between an apparatus for providing a telemedicine service, a patient terminal, and a health care provider terminal.

FIG. 2 illustrates an apparatus for providing a telemedicine service, according to an embodiment.

FIG. 3 is a diagram illustrating a consultation registration process provided by a patient client on a patient terminal, according to an embodiment.

FIGS. 4 to 6 are examples of a screen on which content is provided to a patient entering a virtual consultation room, according to embodiments.

FIG. 7 is a diagram illustrating consultation priority and consultation order adjustment, according to an embodiment.

FIG. 8 is a diagram illustrating the start of consultation, according to an embodiment.

### BEST MODE FOR CARRYING OUT THE INVENTION

Hereinafter, the embodiments will be described in detail with reference to the accompanying drawings. The scope of the right, however, should not be construed as limited to the embodiments set forth herein. In the drawings, like reference numerals are used for like elements.

The terms used herein are selected from terms generally understood by those skilled in the related art, but may have different meanings according to technical developments and/or changes, practices, and preferences of an engineer. Accordingly, the terms used herein should not be construed as limiting the technical spirit, and should be construed as illustrative terms to describe embodiments.

In addition, in a specific case, most appropriate terms are arbitrarily selected by the applicant. In this instance, the meanings of the arbitrarily used terms will be clearly explained in the corresponding description. Hence, the terms should be understood not by the simple names of the terms but by the meanings of the terms and the following overall description of this specification.

An apparatus for providing a telemedicine service according to an embodiment may be an electronic device. The electronic device may include a general-purpose computing device and the like implemented by hardware and/or application software in the form of a system on a chip (SoC), as long as the electronic device provides a service connection and implementation.

For example, the electronic device may include at least one of a smartphone, a tablet personal computer (PC), a mobile phone, a video phone, an e-book reader, a desktop PC, a laptop PC, a netbook computer, a personal digital assistant (PDA), a portable multimedia player (PMP), an MP3 player, a mobile medical device, a camera, or a wearable device (e.g., a head-mounted-device (HMD) such as electronic glasses, an electronic garment, an electronic bracelet, an electronic necklace, an electronic appcessory, an electronic tattoo, or a smartwatch).

According to an additional embodiment, the electronic device may include at least one of various medical devices (e.g., magnetic resonance angiography (MRA), magnetic resonance imaging (MRI), computed tomography (CT), an imaging device, a ultrasound device, etc.), a navigation device, a global positioning system (GPS) receiver, an event data recorder (EDR), a flight data recorder (FDR), a vehicle infotainment device, an electronic device for vessel (e.g., a marine navigation system and a gyro compass, etc.), avionics, a security device, a vehicle head unit, an industrial or household robot, an automatic teller's machine (ATM) of a financial institution, or a point of sales (POS) of a hospital.

In addition, the electronic device may include at least one of a portion of a building/structure or intelligent furniture for a telemedicine service connection, an electronic board, an electronic signature receiving device, a projector, or various measuring instruments (e.g., water, electricity, gas, or radio wave measuring instruments, etc.). The electronic device according to various embodiments of the present disclosure may be one of the various devices described above or a combination thereof. In addition, the electronic device according to various embodiment of the present disclosure may be a flexible device. It is obvious to one having ordinary skill in the art that the type of the electronic device may be various and the type and main purpose (other than the telemedicine service provision function) of the electronic device are not limited the descriptions provided above.

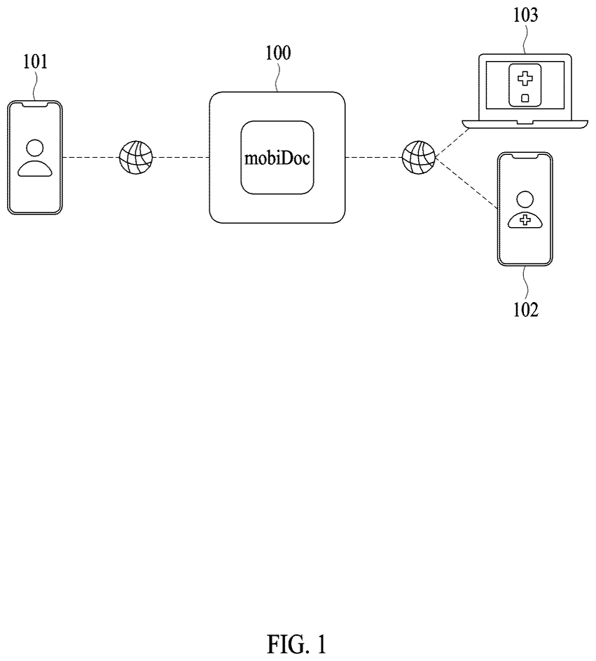

FIG. 1 is a diagram illustrating a connection between an apparatus for providing a telemedicine service, a patient terminal, and a health care provider terminal.

Referring to the overview diagram, an apparatus 100 for providing a telemedicine service may be implemented by a server-class computer, a workstation, a desktop computer, a laptop computer, a mobile electronic device, and the like and may also be implemented by a plurality of devices among the listed devices.

The apparatus 100 may be connected to a patient terminal 101 via a network and may also be connected to a health care provider terminal 102 and a hospital terminal 103. Each of the terminals 101 to 103 may correspond to a mobile electronic device and the like, such as a desktop computer, a laptop computer, a smartphone, and a tablet PC. Each of the terminals (e.g., 101 to 103) may include a software means, which is referred to as a “client” for convenience.

A patient client may be executed on the patient terminal 101. A health care provider client may be executed on the health care provider terminal 102, and a hospital client may be executed on the hospital terminal 103. Here, the hospital client may be a means by which administrative staff or nurses, rather than health care providers (doctors) conducting consultations, perform tasks such as patient registration and billing. While an example is stated where a hospital client exists separately from a health care provider client, in some embodiments, these two clients may be integrated and operated as a single entity.

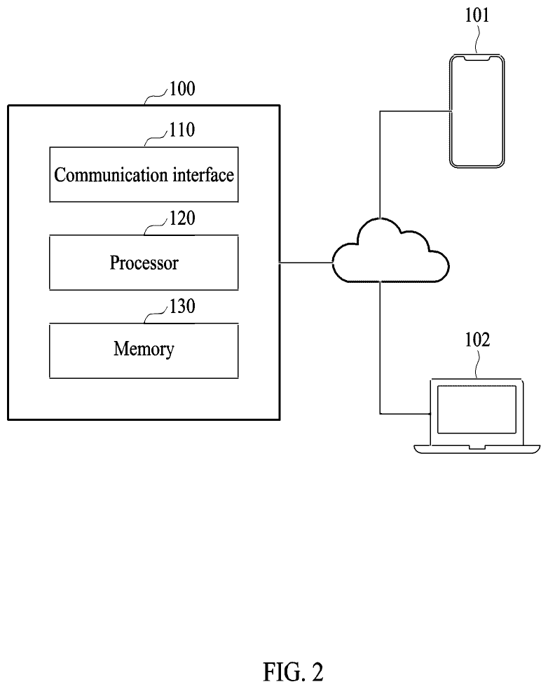

A client installed on the terminals 101 to 103 may be original application software that is installed, a service provided through a page accessed via a web-based browser, or a hybrid app that combines the characteristics of both an app and a web. Since such implementations are feasible for developers with ordinary skill in the art, further detailed description thereof is omitted. Even when embodiments describe one example, it does not exclude other implementations. The implementation and operation of the apparatus 100 is described with reference to FIG. 2.

FIG. 2 illustrates the apparatus 100 for providing a telemedicine service, according to an embodiment.

Ensuring a Health Care Provider's Immediate Availability for a Consultation by Confirming that a Patient is Waiting in a Virtual Consultation Room

The apparatus 100 may include a wired and/or communication interface 110 for communication via a network and a processor 120. The processor 120 may be a general-purpose or specialized hardware resource used in a modern computing device, such as a central processing unit (CPU), a graphics processing unit (GPU), an application processor (AP), and a field programmable gate array (FPGA). The processor 120 may perform operations based on software executed (or provided via the network) on the apparatus 100, and the operations are described in detail.

According to an embodiment, a patient client associated with a patient terminal may complete consultation registration and request access to a virtual consultation room. Then, depending on the approval of a hospital and/or a health care provider, an account of the patient may be admitted to a virtual consultation room stage. This signifies the transition from the consultation registration process to the consultation waiting process. The virtual consultation room is a stage at which, once the health care provider starts a consultation, a video and/or voice communication between the health care provider and the patient may start immediately. This provides a user experience similar to an offline, real-world hospital setting, in which a nurse guides a patient into a consultation room to wait, informs a health care provider that the patient is ready, and the health care provider starts the consultation as soon as they are prepared. In university hospitals, health care providers may alternate between two adjacent consultation rooms. While a health care provider is conducting a consultation in one room, a nurse prepares the next patient in the adjacent room. In this case, an electronic medical record (EMR) system or other means are used to prepare information such as charts, necessary medical images, and test results, ensuring that the busy health care provider does not waste time on these preparatory steps.

The health care provider may finish the consultation with the current patient, complete the necessary charting, and then move to the adjacent room to immediately start the consultation with the prepared patient. This may reduce unnecessary waste of time, allowing the health care provider to stay focused on the essence of the consultation even while seeing the same number of patients. It may also minimize patient inconvenience due to delays in consultation and enables the provision of medical services to a greater number of patients.

The role of the apparatus 100 according to embodiments goes beyond only providing a telemedicine service between the patient terminal 101 and the health care provider terminal 102 and provides better services to both health care professionals and patients by improving consultation efficiency.

As described above, most of the telemedicine support services introduced in the past involve a patient applying for an online (remote) consultation, after which a health care provider (or hospital) attempts to connect with the patient. This connection may be through a video call, a voice call, and the like. However, since telemedicine occurs online and does not involve direct face-to-face interaction, a patient may engage in other activities or leave a terminal unattended and go elsewhere while waiting for the remote consultation. As a result, when the health care provider (or hospital) is ready to start the consultation, the immediate consultation availability may not be guaranteed.

According to an embodiment, the processor 120 may check through the patient client whether the patient is actually ready for the consultation and if the health care provider may start the consultation procedure immediately. The results of this check may be made available to the health care provider through the health care provider terminal 102. According to an embodiment, the processor 120 may process a video connection request from the health care provider client on the health care provider terminal 102 when the patient client on the patient terminal 101 is connected to a virtual consultation room and maintains the connection with the virtual consultation room.

When the result of the checking of the patient client indicates that the patient is on a call, not staring at the screen, away from the device, or engaged in other activities on the terminal 101, thereby causing a delay when the health care provider attempts to start the consultation, the health care provider may be informed of this situation. Furthermore, not only just notifying the health care provider, the processor 120 may also remind the health care provider that the patient may not be ready for the consultation and provide the option to examine another patient first.

With this consideration, the health care provider may know which patients among patients who have registered and entered the virtual consultation room are ready to start a consultation. The health care provider may then select a well-prepared patient to start a consultation, thereby avoiding inefficiencies caused by connection delays or failures. For a patient who is not ready, hospital staff or a telemedicine service provider may induce the patient to be ready (to not close the app, refrain from other activities, and keep an eye on the virtual consultation room screen while waiting) for a consultation by notifying the patient that the patient needs to be prepared on the patient terminal to start a consultation. According to the embodiments, the likelihood that the health care provider may immediately start a consultation with the patient increases.

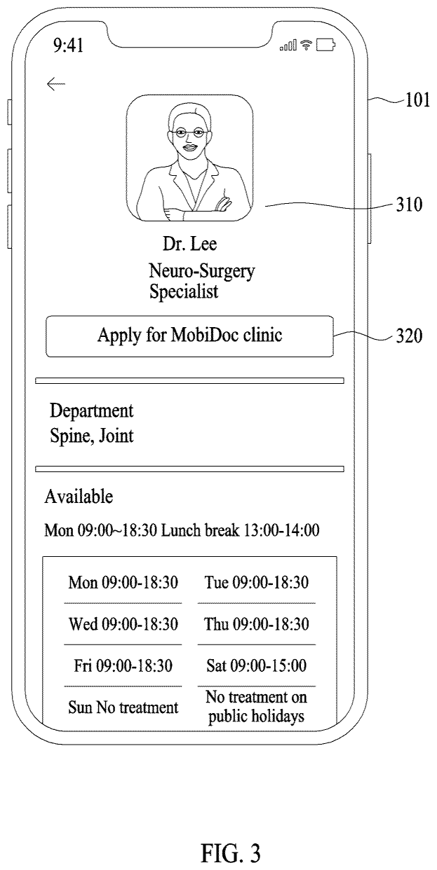

FIG. 3 is a diagram illustrating a consultation registration process provided by a patient client on the patient terminal 101, according to an embodiment. A patient who reviews information 310 such as the name, photo, and qualification profile of a health care provider may select an “apply for telemedicine” button 320, and then a consultation request may be transmitted to a hospital client and/or a health care provider client.

Additionally, the patient may enter necessary information for the consultation. The necessary information may include symptoms, payment method settings, contact details, and the like. Once this process is completed, the consultation registration is finalized. A patient client then may be in a state of waiting for approval from the hospital and/or health care provider. When approval is granted, the patient is not immediately connected to the health care provider but is instead requested to enter a “virtual consultation room.” This entry request may be implemented through various techniques, such as a message popup on the patient client, messaging via a social network service (SNS), texting through communication services, or a push notification on the terminal 101.

“Entering a virtual consultation room” is different from simply registering for a consultation, as entering the virtual consultation room is started by approval (or an invitation to enter) from the health care provider client or hospital. Therefore, once the consultation registration is completed, the patient does not have to wait indefinitely in the virtual consultation room for the health care provider. From the patient's perspective, it is inconvenient to wait without knowing how long it will take for the health care provider to start the consultation. Thus, through the “entering a virtual consultation room” request procedure, the patient may stop other activities and appropriately wait for the health care provider to start a consultation when the consultation is about to start. From the health care provider's perspective, a patient who enters the virtual consultation room is highly likely to be ready for an immediate consultation, making the process convenient for both parties. When a patient who is requested to enter the virtual consultation room does not enter or when the patient enters but is not ready by engaging in other activities instead of waiting for the consultation to start, the health care provider may recognize this situation in advance and postpone the consultation with that patient to see another patient. As a result, the likelihood of failed consultation is significantly reduced compared to the conventional methods.

The following embodiments will describe various methods of determining whether a patient remains ready after entering a virtual consultation room and notifying a health care provider client accordingly.

### Monitoring Connection Maintenance Through a Patient Client

According to an embodiment, the processor 120 may repeatedly check whether the patient client maintains the connection to the virtual consultation room and provides the results of this check to the health care provider client. As described above, the health care provider may then start a consultation with a patient who maintains the connection to the virtual consultation room.

For example, but without being limited thereto, in order to monitor the connection maintenance of the patient client, the processor 120 may use HTML5-based WebSocket to exchange a signal with the patient client through the communication interface 110. Through this process, it may be determined whether the patient client is maintaining the connection based on whether the patient client is providing a predetermined response in the virtual consultation room. When remote medical service software on the patient terminal 101 is closed, the patient client exits the virtual consultation room, or the patient is not waiting for the health care provider to start the consultation for any reason, this information may be communicated to the health care provider and the hospital.

### Checking Whether a Patient is Staring at a Camera of a Terminal to Confirm Readiness for a Consultation

According to an embodiment, the processor 120 may check, through the patient client that enters the virtual consultation room, whether a person who is staring at a camera associated with the patient terminal 101 is identified by the camera. Then, the processor 120 may provide the result of this check to the health care provider client and/or hospital client. Typically, the patient client is a personal device such as the smartphone, tablet, or PC of a patient. Therefore, when someone is staring at the camera of the personal device, it may indicate that the patient is still in the virtual consultation room after entering the virtual consultation room and is a prepared patient waiting for the health care provider to start the consultation.

Therefore, in this embodiment, the processor 120 may continuously check, through the patient client entering the virtual consultation room, whether a person who is staring at the camera associated with the patient terminal is identified by the camera.

For example, without being limited thereto, the processor may utilize the MobileNets model. This model may be used in an embedded vision app, may be based on a depth-wise separable convolutions structure, and may have two simple hyper-parameters. These two hyper-parameters may be appropriately selected based on the environment in which the hyper-parameters are used, and the size of the hyper-parameters may also be selectively applied.

For example, the processor 120 may continuously check whether a face is being recognized by the camera of the patient terminal 101 associated with the patient client using a face recognition open source, face-API, which utilizes the MobileNets ver. 1 model. The result of this check may be provided as a value between 0 and 1, with a value closer to 1 indicating that a face is being recognized.

According to an embodiment, a predetermined threshold may be set, and when the result value is equal to or greater than this threshold, it may be determined that the patient is staring at the camera in the virtual consultation room of the patient client and waiting for the consultation. When the result value remains below the threshold for a predetermined period of time, for example, more than 5 seconds, it may be determined that the patient is not waiting in the virtual consultation room of the patient client. This information may be then provided to the hospital or health care provider client, indicating that the patient is not waiting in the virtual consultation room. Therefore, the health care provider may avoid connecting (transmitting a consultation start request) to a non-waiting patient who has a high probability of failing to start a consultation immediately.

According to an embodiment, when the determined value is less than the predetermined threshold, a request to stare at a screen and wait for a consultation may be provided to the patient through the patient client. This request may be executed via a popup during app operation, message transmission through an SNS, texting, system alarms, push notifications, and the like. Such a request may also be made through voice output on the patient terminal 101. This may be a process similar to how audio continues to play even when another app is executed during a voice or video call on a smartphone.

Meanwhile, according to another embodiment, the processor 120 may selectively and/or concurrently use different techniques to verify whether the patient is ready for the consultation in the virtual consultation room. This involves providing content through the patient client in the virtual consultation room and monitoring an event in which the patient interfaces with the patient client. This will be described in detail with reference to the drawings.

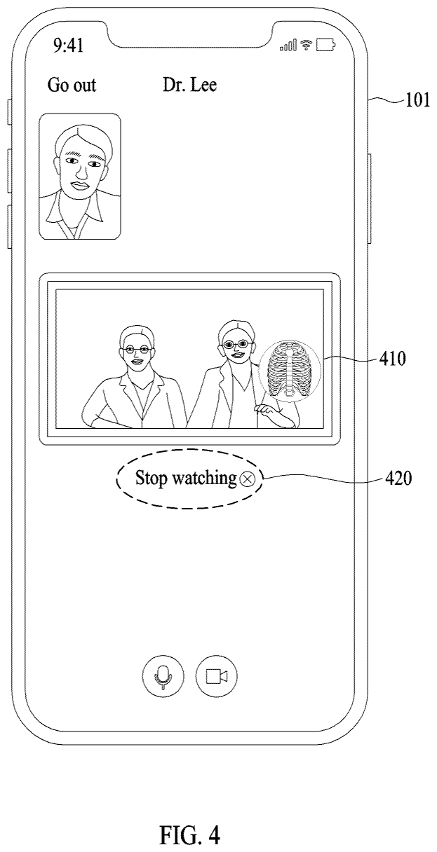

FIGS. 4 to 6 are examples of a screen on which content is provided to a patient entering a virtual consultation room, according to embodiments.

### Monitoring an Event Through a Patient Client

When a health care provider intends to start a telemedicine consultation with a patient in the virtual consultation room, other embodiments are provided to ensure the patient is prepared. According to an embodiment, the processor 120 may provide content on the screen of the virtual consultation room when a patient client maintains a connection to the virtual consultation room.

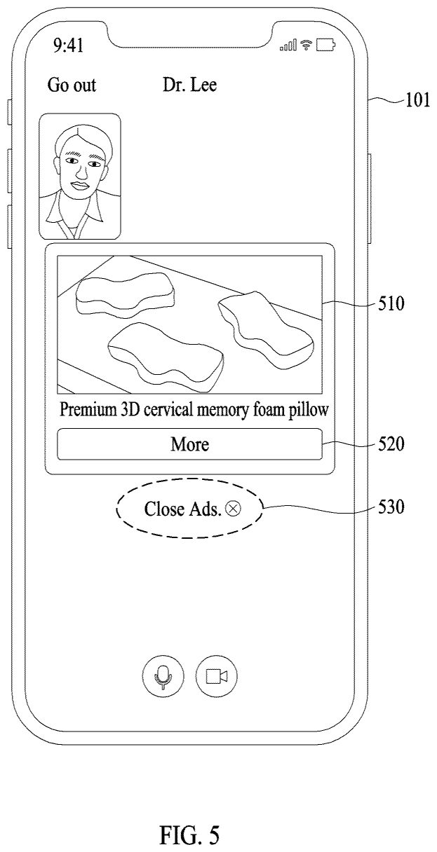

The content may be media content selected by a health care provider or hospital. For example, the provided content may include a YouTube video (410 in FIG. 4) featuring a health care provider, a promotional photo or video (not shown) about a hospital or health care provider, informational content (not shown) about diseases or treatment, and advertisement content for medical devices, supplies, or pharmaceuticals (510 in FIG. 5). However, these are only examples, and it shall not be construed as being limited to the examples described above.

According to an embodiment, it may be detected whether an event occurs in which the patient interfaces with the provided content through the patient client, and the detection result may be provided to the health care provider client. For example, without being limited thereto, the event may be an input (close) to stop the provided content. Examples may include “Stop watching” 420 illustrated in FIG. 4 or “Close Ads” 530 illustrated in FIG. 5. When the patient selects to stop the content, it means that at least at that moment, the patient is watching the content provided in the virtual consultation room. Therefore, at this time, when the health care provider starts the consultation, there is a high probability that the health care provider meets the patient face-to-face immediately.

Embodiments not only provide content to help a patient use the wait time in the virtual consultation room effectively but also determine whether the patient is still prepared for the consultation by detecting whether the patient inputs anything through event monitoring. This is because without such event monitoring, it may be difficult to confirm whether the patient has left the terminal 101 and gone elsewhere during the provision of videos or advertisements, thus being unprepared for the start of the consultation.

Such event monitoring may also include a playback control input (e.g., play, pause, or forward/backward navigation) for video content 410 presented in FIG. 4. Additionally, event monitoring may include the selection (e.g., playing a recommended video, clicking/tapping on a purchase link for an advertised item, etc.) of an additional link 520 associated with advertisement content 510 presented in FIG. 5.

Such an event may be registered with an event listener, which may be optionally added or removed to or from the event listener. The event listener may be managed in a separate database (not shown), and access to the event listener may be achieved through processing by the processor 120 with the assistance of the memory 130.

### Another Example of Monitoring a Patient's Readiness for a Consultation

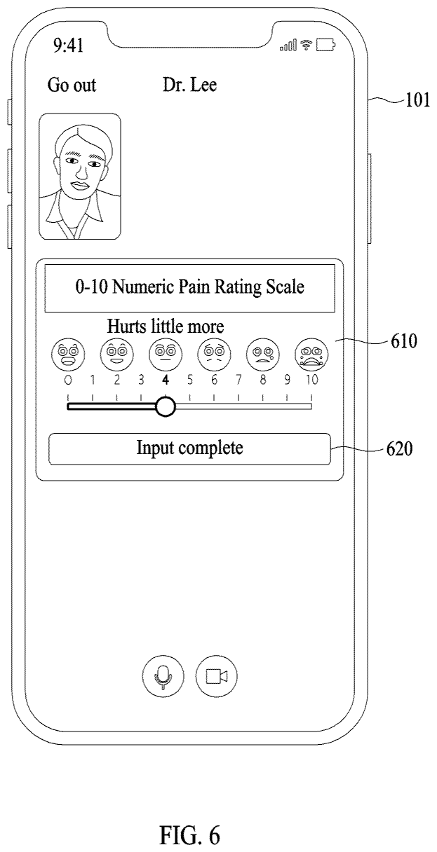

When a health care provider intends to start a telemedicine consultation, an additional embodiment is provided to ensure a patient is prepared. According to the embodiment shown in FIG. 6, when a patient client maintains the connection to a virtual consultation room on the patient terminal 101, the processor 120 may request information input through the patient client, such as inputting a pain scale score (VAS) 610. In addition to the pain scale score, other possible inputs may include information on medication side effects (not shown), responses to a survey about past consultations (not shown), questions the patient desires to ask the health care provider (not shown), photos of the affected area (not shown) of the patient, or descriptions of the symptoms (not shown) of the patient.

As described with reference to FIGS. 4 and 5, when a user responds to such input requests and provides some information, it serves as an indirect means of confirming that the patient is waiting in the virtual consultation room for the consultation to start.

Additionally, providing such information in advance may help reduce consultation time and improve the quality of the consultation by allowing the health care provider to obtain information that may typically be gathered during the consultation. According to this embodiment, the processor 120 may notify the health care provider whether the patient is in the process of inputting the requested information (i.e., whether the patient is ready for the consultation). Furthermore, the processor 120 may also provide the health care provider client with a quantitative assessment of how much of the required information is input.

When there are multiple patients inputting information in response to the requests, the health care provider may select to start the consultation with a patient who is ready first, for example, the patient who presses “Input complete” 620. Even when the information input is not entirely finished, if the health care provider requests to start the consultation with a patient who is in the process of inputting information, the health care provider may be assured of a high probability of being able to start the consultation immediately.

Meanwhile, a health care provider providing a telemedicine consultation often also sees a patient who visits the hospital in person. Therefore, the apparatus 100 for providing a telemedicine service may not only assist with providing a telemedicine service but also help ensure that a telemedicine service is seamlessly integrated with the care of a patient who visits the hospital.

### Evaluating Readiness for a Waiting Patient and Providing a Possibility of Selecting a Target to be Treated

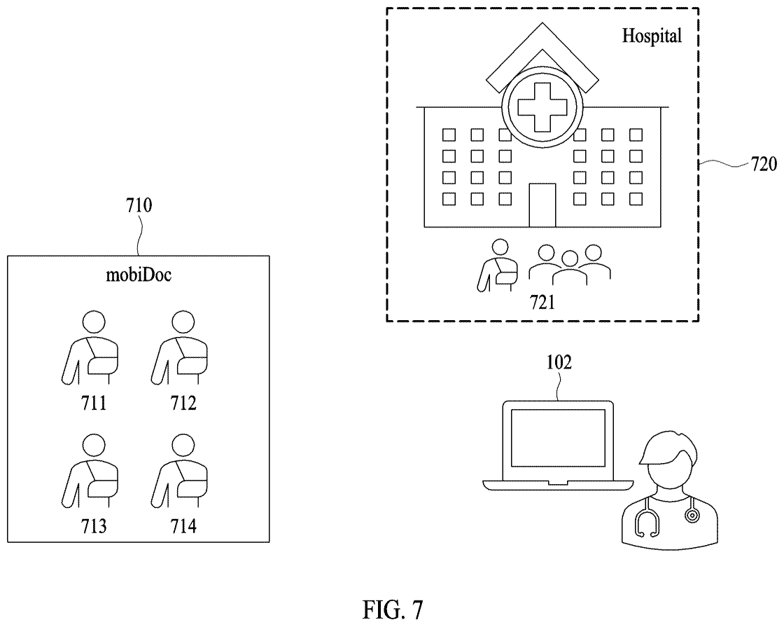

FIG. 7 is a diagram illustrating consultation priority and consultation order adjustment, according to an embodiment.

According to an embodiment, when a plurality of different patient clients 711, 712, 713, and 714 simultaneously maintains a connection to a virtual consultation room, the processor 120 may evaluate the readiness for a consultation of each patient client individually and provide the results to a health care provider client.

In actual medical settings, a patient who registers for a consultation first or has an earlier appointment time usually sees a health care provider first. However, in the case of online connections, a patient who has registered for a consultation and is waiting in a virtual consultation room may sometimes be less prepared or not prepared at all. Therefore, in this embodiment, the apparatus for providing a telemedicine service may not process a patient consultation on a first-in first-out (FIFO) basis but allow the health care provider or hospital to identify which of the patient clients 711 to 714 of the patients waiting in the virtual consultation room is ready first or has completed the necessary input for the consultation, thereby improving consultation efficiency. This may also be compared with patients 721 who are waiting in a hospital 720, and the health care provider may view both the health care provider client and the internal information processing system of the hospital, seamlessly switching between telemedicine and in-person consultations. Patients waiting for telemedicine consultations are mixed with patients waiting in the hospital for their names to be called. Through this process, telemedicine integrates smoothly with the flow of in-person patient consultations without disrupting in-person patient consultations, and thus the health care provider may naturally treat patients connected via telemedicine as if they are in-person patients, rather than having a disparate experience.

According to an embodiment, the process of the processor evaluating the readiness of individual patient clients may be based on scoring various evaluation items. For example, the readiness of a first patient client may be determined using at least one piece of information of whether a person who stares at a camera associated with the first patient client is recognized by the first patient client, whether input information requested from the first patient client is input, the priority according to a predefined consultation priority for the first patient client, consultation compliance evaluated based on the existing average consultation time for the first patient client, or whether the first patient client is a chronic disease patient.

### Informing a Waiting Patient of an Estimated Wait Time

According to an embodiment, when admitting the patient client to the virtual consultation room, the processor 120 may provide an estimated wait time. Therefore, a patient waiting for a telemedicine consultation may have some idea of how long the patient needs to wait. Embodiments ensure that whenever the health care provider is ready to start the consultation, the patient is waiting in the virtual consultation room and may immediately face the health care provider on the screen. Therefore, the intent is not to encourage the patient client to disconnect the patient client or switch the patient client to another screen during the estimated wait time.

Since the patient is already in the virtual consultation room, the patient may be allowed to predict the wait time and be encouraged to stay focused on the patient client in the virtual consultation room. This process may involve providing content or requesting information input, as described with reference to FIGS. 4 to 6. The patient needs to remain in the virtual consultation room screen while waiting for the health care provider to start the consultation, ensuring that the patient stays attentive to the provided content.

According to an embodiment, when calculating the estimated wait time, the processor may use at least one of the number of other patient clients already admitted to the virtual consultation room of the same health care provider or the number of patients waiting in the hospital for an in-person consultation with the same health care provider.

In this case, the estimated wait time may reflect the sum of a first time, which is the product of a previous average telemedicine consultation time of the health care provider and the number of patients currently waiting for a telemedicine consultation, and a second time, which is the product of a previous average consultation time of the health care provider and the number of patients currently waiting in the hospital for the consultation by the health care provider.

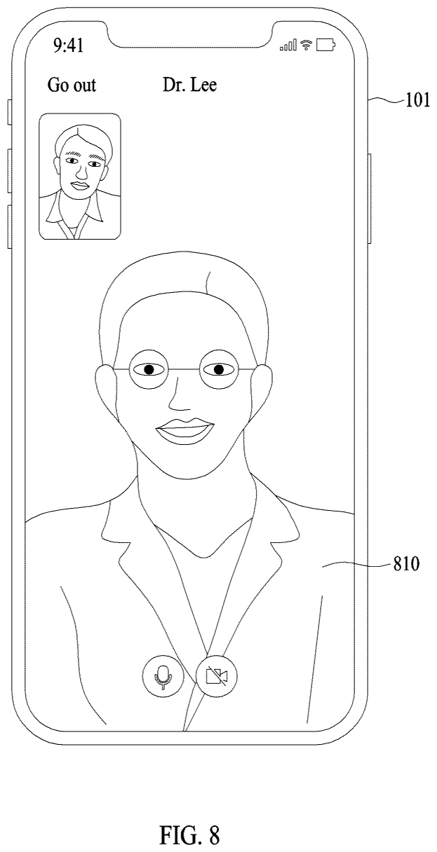

FIG. 8 is a diagram illustrating the start of consultation, according to an embodiment.

When a health care provider 810 starts a consultation, a patient waiting in a virtual consultation room may immediately start the consultation by facing the health care provider on the screen of the terminal 101.

The units described herein may be implemented using a hardware component, a software component and/or a combination thereof. A processing device may be implemented using one or more general-purpose or special purpose computers, such as, for example, a processor, a controller and an arithmetic logic unit (ALU), a digital signal processor (DSP), a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or one or more combinations thereof, to independently or uniformly instruct or configure the processing device to operate as desired. Software and data may be embodied permanently or temporarily in any type of machine, component, physical or pseudo equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and/or DVDs; magneto-optical media such as floptical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter. The hardware devices described above may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and equivalents to the claims are also within the scope of the following claims.

1. An apparatus for providing a telemedicine service, the apparatus comprising:
a communication interface; and a processor,
wherein the processor is configured to start a video connection between a health care provider client and a patient client by admitting the patient client into a virtual consultation room when the patient client associated with a patient terminal completes medical consultation registration and requests access to the virtual consultation room and processing a video connection of the health care provider client associated with a health care provider terminal when the patient client is connected to the virtual consultation room.

2. The apparatus of claim 1, wherein the processor is configured to repeatedly check whether the patient client maintains the connection to the virtual consultation room and provide the health care provider client with information on whether the connection is maintained.

3. The apparatus of claim 2, wherein the processor is configured to exchange a signal with the patient client through the communication interface using HTML5-based WebSocket and determine whether the connection is maintained based on whether the patient client provides a predetermined response in the virtual consultation room.

4. The apparatus of claim 1, wherein the processor is configured to provide the health care provider client with information on whether a person staring at a camera associated with the patient terminal through the patient client is identified by the camera.

5. The apparatus of claim 1, wherein the processor is configured to provide content on a screen of the virtual consultation room when the patient client maintains the connection to the virtual consultation room and provide the health care provider client with information on whether an event associated and interfacing with the content occurs in the patient client.

6. The apparatus of claim 5, wherein the event is a user input corresponding to at least one of an input to stop providing the content, an input to control playback of the content, or selection of an additional link for the content and is registered with an event listener.

7. The apparatus of claim 1, wherein, when the patient client maintains the connection to the virtual consultation room, the processor is configured to transmit an input request asking for at least one of a pain scale score (visual analogue scale (VAS)), medication side effect information, a response to a survey related to past consultation, a photo of an affected area of a patient, or a description of a symptom that a patient has and provide the health care provider client with information on whether the patient client is responding to the input request.

8. The apparatus of claim 1, wherein
the processor is configured to, when a plurality of different patient clients simultaneously maintains a connection to the virtual consultation room, evaluate consultation readiness of each patient client and provide the health care provider client with a result of the evaluation, and
the readiness of a first patient client of the plurality of different patient clients is determined using at least one piece of information of:
whether a person staring at a camera associated with the first patient client is recognized, whether input information requested from the first patient client is completed, priority based on a predetermined criterion for consultation priority for the first patient client, consultation compliance evaluated according to an existing average consultation time for the first patient client, or whether the first patient client is a chronic disease patient.

9. The apparatus of claim 1, wherein, when admitting the patient client into the virtual consultation room, the processor is configured to provide an estimated wait time, and the estimated wait time is calculated using at least one of a number of other patient clients already in the virtual consultation room or a number of patients waiting in a hospital for a consultation by a health care provider using the health care provider client.

10. The apparatus of claim 9, wherein the estimated wait time is calculated by reflecting a sum of a first time, which is a product of a previous average telemedicine consultation time of the health care provider and a number of early-registered patients currently waiting for telemedicine, and a second time, which is a product of a previous average consultation time of the health care provider and a number of patients currently waiting in the hospital for consultation by the health care provider.
